(19) United States
(12) Patent Application Publication
(10) Pub. No.: US 2025/0325723 A1
(43) Pub. Date: Oct. 23, 2025

(54) CREATIVE ORNAMENT WITH SMOKE EFFECT

(71) Applicant: Juanjuan HUANG, Shaoyang (CN)

(72) Inventor: Juanjuan HUANG, Shaoyang (CN)

(21) Appl. No.: 19/257,491

(22) Filed: Jul. 2, 2025

(51) Int. Cl. A61L 9/03

(52) U.S. Cl. CPC A61L 9/03

(57) ABSTRACT

A creative ornament with a smoke effect is provided, including an ornament main body. A placement position of a smoke generation element is arranged on the ornament main body. A guide structure is arranged at the placement position. The guide structure is configured to guide smoke to flow downwards. A creative ornament with a smoke effect is further provided, including an ornament main body and an incense. A placement position is arranged on the ornament main body. The incense is arranged at the placement position. A guide structure is arranged at the placement position. The guide structure is configured to guide smoke to flow downwards.

### TECHNICAL FIELD

The present disclosure relates to the field of creative ornaments, and in particular, to a creative ornament with a smoke effect.

### BACKGROUND

With the improvement of the living standard of people, after material needs are met, spiritual needs also need to be met. For example, in order to enhance the sense of beauty of a living or working environment, various decorative items are arranged in the environment, and creative ornaments are commonly used. At present, an existing creative ornament mainly focuses on a creative shape, usually with a stationary structure, which limits a presentation effect. Moreover, the existing creative ornament has low decorativeness and lacks sense of beauty. This further limits its usage scenario and decorative effect.

### SUMMARY

In order to overcome the shortcomings in the prior art, the present disclosure provides a creative ornament with a smoke effect.

Technical solutions used in the present disclosure to solve the technical problems are as follows:

The present disclosure provides a creative ornament with a smoke effect, including an ornament main body. A placement position for a smoke generation element is arranged on the ornament main body. A guide structure is arranged at the placement position. The guide structure is configured to guide smoke to flow downwards.

The guide structure is configured to guide the smoke to flow downwards along the ornament main body.

The guide structure is arranged at a bottom of the placement position.

The guide structure has a through portion. The through portion is configured to guide the smoke to flow downwards.

The through portion extends in a penetrating manner at the placement position from top to bottom.

The guide structure is a guide through hole or a guide through slot.

An upper end portion of the through portion is configured to be in contact with a bottom of the smoke generation element.

A guide slot is provided in a surface of the ornament main body.

The guide slot is located below the through portion, and the guide slot is communicated to a lower end opening of the through portion.

The guide slot is distributed along the surface of the ornament main body.

The guide slot is distributed along the surface of the ornament main body from top to bottom.

An upper end opening of the guide slot is communicated to the lower end opening of the through portion.

A gathering cavity for smoke is provided in a position of the ornament main body close to a bottom.

The guide slot is communicated to the gathering cavity.

A lower end opening of the guide slot is communicated to the gathering cavity.

The placement position is arranged at a position of the ornament main body close to a top.

The present disclosure further provides a creative ornament with a smoke effect, including an ornament main body and an incense. A placement position is arranged on the ornament main body. The incense is arranged at the placement position. A guide structure is arranged at the placement position. The guide structure is configured to guide smoke to flow downwards.

A smoke outlet hole is provided in a bottom of the incense.

The guide structure has a through portion. The through portion extends in a penetrating manner at the placement position from top to bottom, to guide the smoke to flow downwards.

The smoke outlet hole is arranged in a manner of corresponding to an upper end opening of the through portion.

Beneficial effects of the present disclosure are as follows: Through the above structural arrangement, during use, the smoke generation element can be placed at the placement position of the ornament main body. Since the smoke generation element can generate smoke and the guide structure is arranged at the placement position, the guide structure can guide the smoke to flow downwards, and a dynamic decorative effect and a decorative effect of transition from smoke surrounding the ornament main body to mist surrounding the ornament main body can be achieved. The creative ornament is more aesthetically pleasing.

### BRIEF DESCRIPTION OF THE DRAWINGS

For clearer descriptions of the technical solutions according to the embodiments of the present disclosure, the drawings required to be used in the description of the embodiments are briefly introduced below. The drawings in the description below are only some embodiments of the present disclosure, and those skilled in the art that other drawings can be acquired according to the drawings without creative efforts.

The present disclosure will be further explained below in conjunction with the accompanying drawings and embodiments.

FIG. 1 is a schematic structural diagram of the present disclosure;

FIG. 2 is a schematic structural diagram of the present disclosure in different angles;

FIG. 3 is a schematic structural diagram of providing an incense in FIG. 1;

FIG. 4 is a schematic structural diagram of providing an incense in FIG. 2;

FIG. 5 is a schematic structural diagram of providing an incense of the present disclosure in different angles;

FIG. 6 is a sectional view cut away along a center of the incense of FIG. 3; and

FIG. 7 is a schematic structural diagram of an incense.

### DETAILED DESCRIPTION OF THE EMBODIMENTS

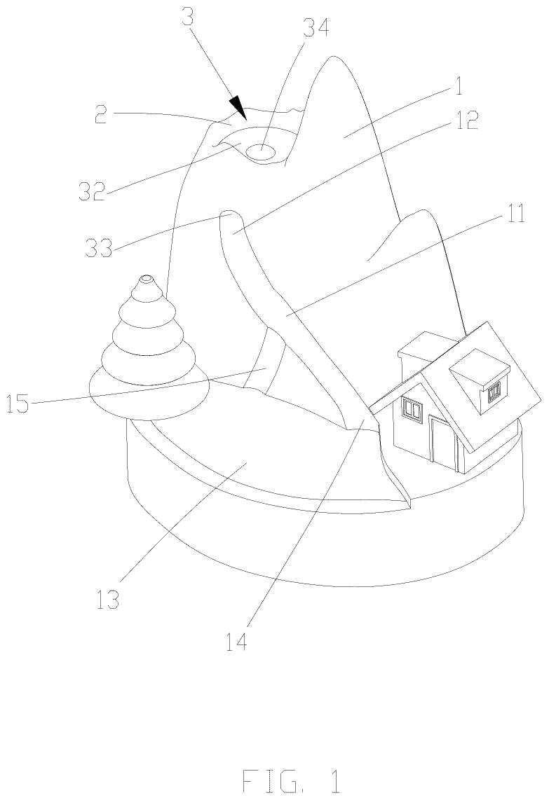

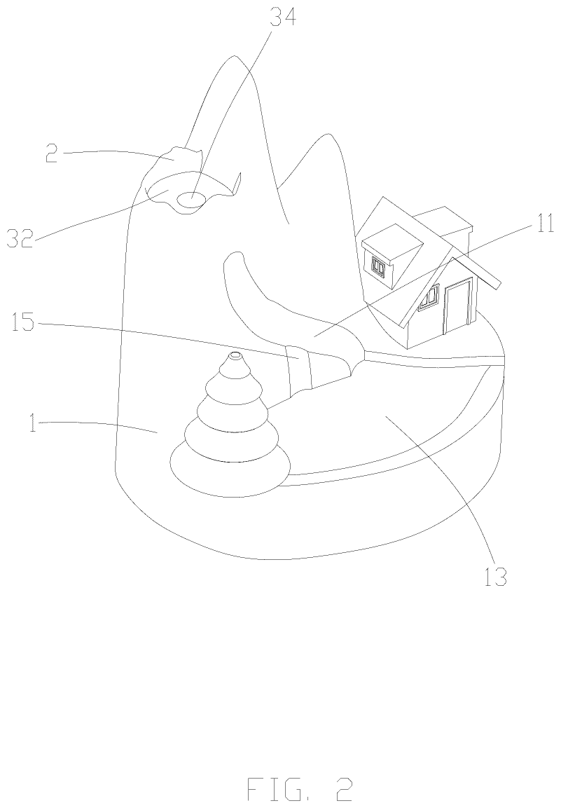

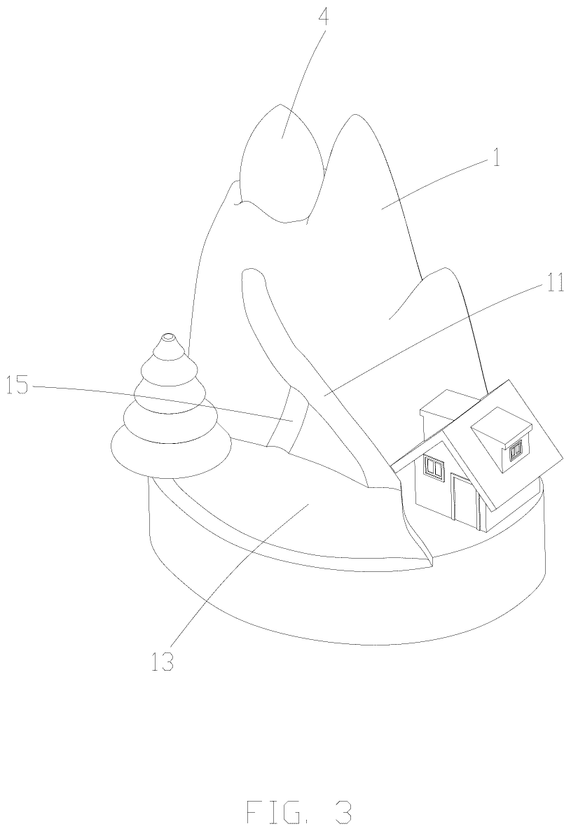

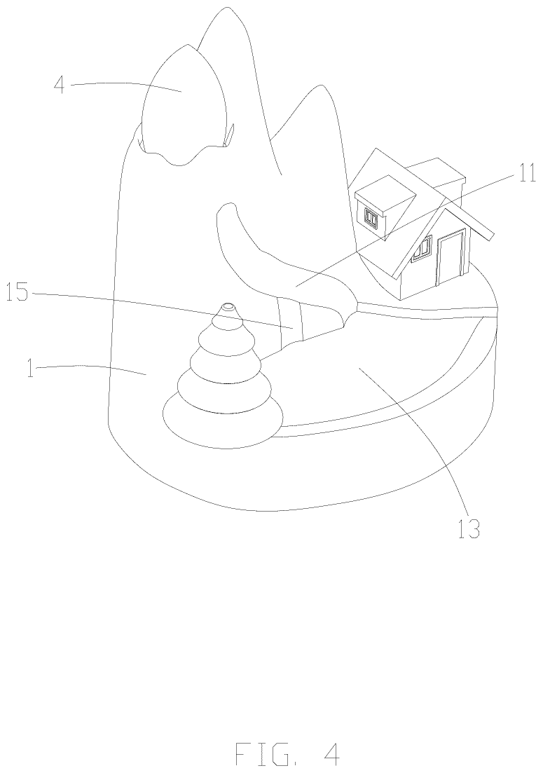

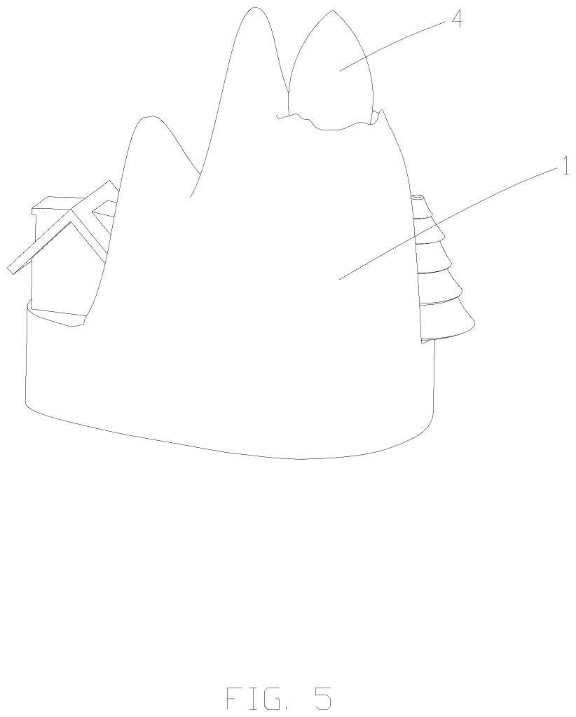

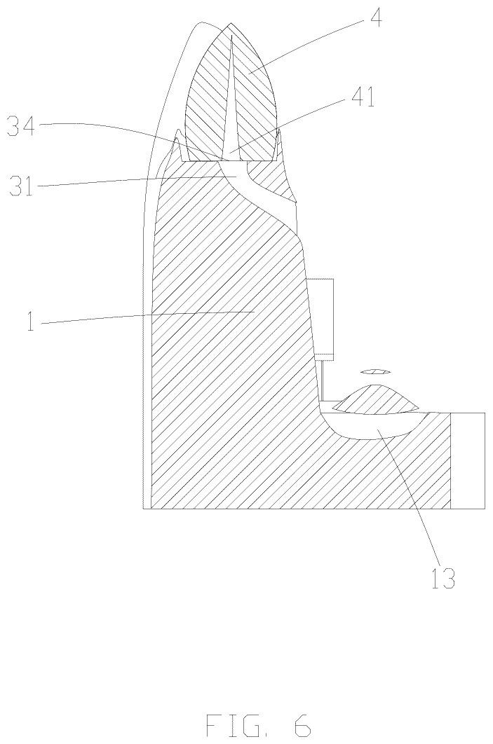

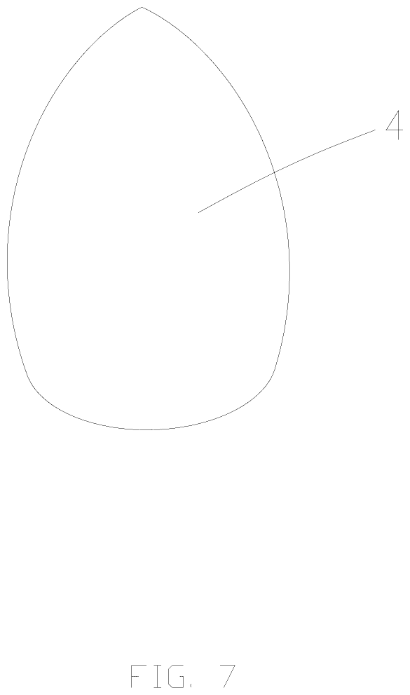

Referring to FIG. 1 to FIG. 7, a creative ornament with a smoke effect includes an ornament main body 1. A placement position 2 for a smoke generation element 4 is arranged on the ornament main body 1. A guide structure 3 is arranged at the placement position 2. The guide structure 3 is configured to guide smoke to flow downwards.

Through the above structural arrangement, during use, the smoke generation element can be placed at the placement position of the ornament main body. Since the smoke generation element can generate smoke and the guide structure is arranged at the placement position, the guide structure can cool the smoke and guide the smoke to flow downwards, and a dynamic decorative effect and a decorative effect of transition from smoke surrounding the ornament main body to mist surrounding the ornament main body can be achieved. The creative ornament is more aesthetically pleasing. The smoke generation element can be an incense, which can generate heavier smoke when burned, to allow the smoke to flow downwards. This type of incense is generally referred to as “backflow incense”. Especially when smoke generated at preliminary combustion is in contact with the guide structure with a low temperature, the guide structure can also cool the smoke, to avoid the smoke from rising along with surrounding hot air and effectively enhance a downward flowing effect of the smoke.

Specifically, the guide structure 3 is configured to guide the smoke to flow downwards along the ornament main body 1, so that the smoke can better cover the ornament main body and have a better dynamic mist surrounding effect.

The guide structure 3 is arranged at a bottom of the placement position 2. Due to the downward flowing of the smoke, the guide structure is arranged at the bottom of the placement position, which can better achieve cooling and guide effects.

The guide structure 3 has a through portion 31. The through portion 31 is configured to guide the smoke to flow downwards. Moreover, the through portion 31 extends in a penetrating manner at the placement position 2 from top to bottom. The structure is simple, and the design is proper.

Further, the guide structure 3 is a guide through hole or a guide through slot. The structure is simple, and the design is proper.

Further, an upper end portion 32 of the through portion 31 is configured to be in contact with a bottom of the smoke generation element 4. Due to the combustion of the incense, air around the incense may have a high temperature, thus generating rising hot air. The contact between the through portion and the bottom of the smoke generation element can avoid the smoke inside the through portion from being affected by the rising hot air, ensure that the smoke inside the through portion flows downwards, and further enhance the guide effect of the through portion.

A guide slot 11 is provided in a surface of the ornament main body 1. Due to the arrangement of the guide slot, the smoke can better flow downwards along the ornament main body.

Further, the guide slot 11 is located below the through portion 31, and the guide slot 11 is communicated to a lower end opening 33 of the through portion 31. Meanwhile, an upper end opening 12 of the guide slot 11 is communicated to the lower end opening 33 of the through portion 31. In this way, the smoke can flow into the guide slot via the lower end opening of the through portion, to achieve a better guide effect.

Further, the guide slot 11 is distributed along the surface of the ornament main body 1. Meanwhile, the guide slot 11 is distributed along the surface of the ornament main body 1 from top to bottom, so that the guide slot can better cover the ornament main body. That is, the smoke can better cover the ornament main body and then have a better dynamic mist surrounding effect.

Further, a gathering cavity 13 for smoke is provided in a position of the ornament main body 1 close to a bottom, so that the downwards flowing smoke can be gathered in the gathering cavity to achieve a better mist effect.

Meanwhile, the guide slot 11 is communicated to the gathering cavity 13. Specifically, a lower end opening 14 of the guide slot 11 is communicated to the gathering cavity 13. The smoke can flow into the gathering cavity via the lower end opening of the guide slot, to achieve a better guide effect.

Further, the guide slot 11 also has a branch slot 15. An upper end opening of the branch slot is communicated to the guide slot, and a lower end opening of the branch slot is communicated to the gathering cavity. The smoke can also flow into the gathering cavity along the guide slot and the branch slot, to enhance the mist effect.

Further, the placement position 2 is arranged at a position of the ornament main body 1 close to a top. The smoke can better cover the ornament main body and then have a better dynamic mist surrounding effect.

The smoke generation element 4 is an incense. Furthermore, the incense can be a cone-shaped incense.

Further, the incense is provided with a smoke outlet hole 41. Furthermore, the smoke outlet hole 41 is provided in a bottom of the incense. The smoke outlet hole can also extend from the bottom of the incense to a top of the incense. Generally, the smoke outlet hole does not penetrate through the top of the incense, to facilitate ignition of the incense. Certainly, the smoke outlet hole can also penetrate through the top of the incense. The smoke outlet hole can be in a conical design. When the incense is combusted, the smoke can flow downwards along the smoke outlet hole. This is beneficial for the downward flowing of the smoke.

Further, the smoke outlet hole 41 is arranged in a manner of corresponding to an upper end opening 34 of the through portion 31. Moreover, a hole diameter of the upper end opening of the through portion is greater than a hole diameter of the smoke outlet hole, so that the incense can be accurately aligned with the upper end opening of the through portion during placement, making it convenient for use. When the incense is combusted, the incense can release smoke downwards, and the smoke can directly flow into the upper end opening of the through portion. This is beneficial for subsequent downward flowing of smoke and the guide effect of the through portion.

In this patent, a proper structural layout of the creative ornament is formed through the above structural arrangement. Specifically, the placement position close to the top of the ornament main body, the guide slot arranged from top to bottom, and the gathering cavity close to the bottom of the ornament main body are formed. During use, the incense is placed at the placement position and is combusted to generate smoke. The smoke enters the upper end opening of the through portion via the smoke outlet hole in the bottom of the incense. Under the guide effect of the through portion, the smoke can flow into the upper end opening of the guide slot through the lower end opening of the through portion. The smoke can then flow along the ornament main body through the guide slot, thereby forming the dynamic mist surrounding effect. Further, the smoke in the guide slot flows into the gathering cavity via the lower end opening of the guide slot, thus forming gathered smoke (visually similar to clouds) and enhancing a mist effect.

Moreover, in this patent, the ornament main body can have various shapes. For example, the ornament main body can be a rockery. Correspondingly, the creative ornament is an ornament in which mists surround mountains. This is more closely related to a decorative effect of natural scenery. The ornament main body can be a figure of Buddha. Correspondingly, the creative ornament is an ornament in which mists surround a figure of Buddha. This has a better decorative effect of a fantasy atmosphere. Certainly, the specific shape of the ornament main body is not limited here, and the above is only an example.

The above shows one or more implementations provided in combination with specific content, and it is not deemed that the specific implementations of the present disclosure is limited to these explanations. Any technical inferences or replacements that are similar or identical to the method and structure of the present disclosure or made under the idea of the present disclosure shall fall within the protection scope of the present disclosure.

What is claimed is:

1. A creative ornament with a smoke effect, comprising an ornament main body (1), wherein a placement position (2) for a smoke generation element (4) is arranged on the ornament main body (1); a guide structure (3) is arranged at the placement position (2); and the guide structure (3) is configured to guide smoke to flow downwards.

2. The creative ornament with the smoke effect according to claim 1, wherein the guide structure (3) is configured to guide the smoke to flow downwards along the ornament main body (1).

3. The creative ornament with the smoke effect according to claim 2, wherein the guide structure (3) is arranged at a bottom of the placement position (2).

4. The creative ornament with the smoke effect according to claim 3, wherein the guide structure (3) has a through portion (31); and the through portion (31) is configured to guide the smoke to flow downwards.

5. The creative ornament with the smoke effect according to claim 4, wherein the through portion (31) extends in a penetrating manner at the placement position (2) from top to bottom.

6. The creative ornament with the smoke effect according to claim 5, wherein the guide structure (3) is a guide through hole or a guide through slot.

7. The creative ornament with the smoke effect according to claim 5, wherein an upper end portion (32) of the through portion (31) is configured to be in contact with a bottom of the smoke generation element (4).

8. The creative ornament with the smoke effect according to claim 7, wherein a guide slot (11) is provided in a surface of the ornament main body (1).

9. The creative ornament with the smoke effect according to claim 8, wherein the guide slot (11) is located below the through portion (31), and the guide slot (11) is communicated to a lower end opening (33) of the through portion (31).

10. The creative ornament with the smoke effect according to claim 9, wherein the guide slot (11) is distributed along the surface of the ornament main body (1).

11. The creative ornament with the smoke effect according to claim 10, wherein the guide slot (11) is distributed along the surface of the ornament main body (1) from top to bottom.

12. The creative ornament with the smoke effect according to claim 11, wherein an upper end opening (12) of the guide slot (11) is communicated to the lower end opening (33) of the through portion (31).

13. The creative ornament with the smoke effect according to claim 12, wherein a gathering cavity (13) for smoke is provided in a position of the ornament main body (1) close to a bottom.

14. The creative ornament with the smoke effect according to claim 13, wherein the guide slot (11) is communicated to the gathering cavity (13).

15. The creative ornament with the smoke effect according to claim 14, wherein a lower end opening (14) of the guide slot (11) is communicated to the gathering cavity (13).

16. The creative ornament with the smoke effect according to claim 13, wherein the placement position (2) is arranged at a position of the ornament main body (1) close to a top.

17. A creative ornament with a smoke effect, comprising an ornament main body (1) and an incense, wherein a placement position (2) is arranged on the ornament main body (1); the incense is arranged at the placement position (2); a guide structure (3) is arranged at the placement position (2); and the guide structure (3) is configured to guide smoke to flow downwards.

18. The creative ornament with the smoke effect according to claim 17, wherein a smoke outlet hole (41) is provided in a bottom of the incense.

19. The creative ornament with the smoke effect according to claim 18, wherein the guide structure (3) has a through portion (31); and the through portion (31) extends in a penetrating manner at the placement position (2) from top to bottom, to guide the smoke to flow downwards.

20. The creative ornament with the smoke effect according to claim 19, wherein the smoke outlet hole (41) is arranged in a manner of corresponding to an upper end opening (34) of the through portion (31).
